(19) United States
(12) Patent Application Publication
(10) Pub. No.: US 2025/0236830 A1
(43) Pub. Date: Jul. 24, 2025

(54) DEVICE FOR OBSERVING MICROORGANISMS

(71) Applicant: NIPPON TELEGRAPH AND TELEPHONE CORPORATION, Tokyo (JP)

(72) Inventors: Manami ITO, Musashino-shi (JP); Sosuke IMAMURA, Musashino-shi (JP); Kazuhiro TAKAYA, Musashino-shi (JP); Kaori TAKAYANAGI, Musashino-shi (JP); Nobuaki ENDO, Musashino-shi (JP)

(73) Assignee: NIPPON TELEGRAPH AND TELEPHONE CORPORATION, Tokyo (JP)

(21) Appl. No.: 18/703,374

(22) PCT Filed: Nov. 18, 2021
(86) PCT No.: PCT/JP2021/042424

§ 371 Date: Apr. 22, 2024

(51) Int. Cl. C12M 1/00; C12N 1/04

(52) U.S. Cl. CPC C12M 23/34; C12M 23/02; C12M 23/22; C12M 29/00; C12M 31/00; C12N 1/04

(57) ABSTRACT

A microorganism observation device capable of continuously culturing and observing microorganisms is provided. A microorganism observation device includes: a main body including a vessel having a hollow structure and a partition wall provided in the vessel and partitioning an internal space of the vessel into compartments, the vessel having one or more inlets and an outlet each communicating the internal space to an external space, the vessel including a light-transmitting portion having light-transmitting properties at positions of the compartments, and the wall allowing transfer of liquid between the compartments and the inlet and between the compartments and the outlet; a medium filling the compartments; and a plurality of types of microorganisms located in the internal space, in which at least one of the compartments includes two or more of the plurality of types of microorganisms and is free from one or more of the plurality of types of microorganisms.

### TECHNICAL FIELD

The present invention relates to a device for observing microorganisms.

### BACKGROUND ART

Unlike microorganisms in water, material diffusion between cell populations in soil microorganisms is restricted by soil particles. For this reason, soil microorganisms are segregated in accordance with relationships such as symbiotic relationships and competitive relationships.

As a method for discovering symbiotic relationships among soil microorganisms, there is a method in which a plurality of arbitrary soil microorganisms are selected and these soil microorganisms are co-cultivated. However, such methods can only be applied to microorganisms which can be isolated and cultured. Also, such methods are very time consuming when observing a large number of combinations composed of a plurality of microorganisms.

As a method for observing a large number of combinations composed of a plurality of microorganisms, for example, NPL 1 describes preparing a plurality of droplets composed of a bacterial liquid including several microorganisms and a lipid membrane surrounding the bacterial liquid, and observing these droplets.

### CITATION LIST

### Non Patent Literature

[NPL 1] Toju, Hirokazu, Kabir G. Peay, Masato Yamamichi, Kazuhiko Narisawa, Kei Hiruma, Ken Naito, Shinji Fukuda, et al., “Core Microbiomes for Sustainable Agroecosystems.” Nature Plants, 4 (5): p. 247-57

### SUMMARY OF INVENTION

An object of the present invention is to provide a device for observing microorganisms capable of continuously culturing and observing microorganisms.

According to a first aspect of the present invention, a device for observing microorganisms includes:
a device main body including a culture vessel having a hollow structure and a partition wall provided in the culture vessel and partitioning an internal space of the culture vessel into a plurality of compartments, the culture vessel having one or more inflow holes and an outflow hole each communicating the internal space to an external space, the culture vessel having a light-transmitting portion having light-transmitting properties at positions of the compartments, and the partition wall allowing transfer of liquid between each of the plurality of compartments and the one or more inflow holes and between each of the plurality of compartments and the one or more outflow holes;
a medium filling each of the plurality of compartments; and
a plurality of types of microorganisms located in the internal space,
in which at least one of the plurality of compartments includes two or more of the plurality of types of microorganisms and is free from one or more of the plurality of types of microorganisms.

According to a second aspect of the present invention, a method for producing device for observing microorganisms, includes:
preparing a culture vessel including an internal space, one or more inflow holes and an outflow hole each communicating the internal space to an external space, and the culture vessel having a light-transmitting portion having light-transmitting properties;
supplying a microorganism-containing liquid containing a plurality of types of microorganisms, a photocurable resin, and a medium to the internal space; and
forming a partition wall made of a cured product of the photocurable resin and partitioning the internal space into a plurality of compartments by irradiating a part of the microorganism-containing liquid with light through the light-transmitting portion so that transfer of a liquid is allowed between each of the plurality of compartments and the one or more inflow holes and between each of the plurality of compartments and the outflow hole, and so that at least one of the plurality of compartments includes two or more of the plurality of types of microorganisms and is free from one or more of the plurality of types of microorganisms.

According to a third aspect of the present invention, a method of culturing microorganisms includes: continuously or intermittently supplying the medium to the device for observing microorganisms according to the first aspect through the one or more inflow holes to culture the plurality of types of microorganisms.

According to a fourth aspect of the present invention, a method of observing microorganisms includes:
observing microorganisms located in one or more of the plurality of compartments through the light-transmitting portion, the microorganisms are two or more of the plurality of types of microorganisms included in the device for observing microorganisms according to the first aspect.

According to the present invention, a device for observing microorganisms which can continuously culture and observe microorganisms is provided.

### BRIEF DESCRIPTION OF DRAWINGS

FIG. 1 is a schematic diagram showing an example of a top view of a device for observing microorganisms according to an embodiment.

FIG. 2 is a cross-sectional view of the device for observing microorganisms shown in FIG. 1 along line II-II.

FIG. 3 is an enlarged view of the device for observing microorganisms shown in FIG. 1.

FIG. 4 is a cross-sectional view showing an example of a method for producing a device for observing microorganisms according to an embodiment.

FIG. 5 is a schematic diagram showing an example of a top view of a device for observing microorganisms according to another embodiment.

FIG. 6 is a schematic diagram showing an example of a top view of a device for observing microorganisms according to still another embodiment.

FIG. 7 is a schematic diagram showing an example of a top view of a device for observing microorganisms according to still another embodiment.

FIG. 8 is a schematic diagram showing an example of a droplet generation device.

### DESCRIPTION OF EMBODIMENTS

Embodiments of the present invention will be described below with reference to the drawings. In the drawings referred to below, the same constituent elements are denoted by the same reference numerals and overlapping descriptions are omitted. Note that the present invention is not limited to the following embodiment.

### 1. Device for Observing Microorganisms

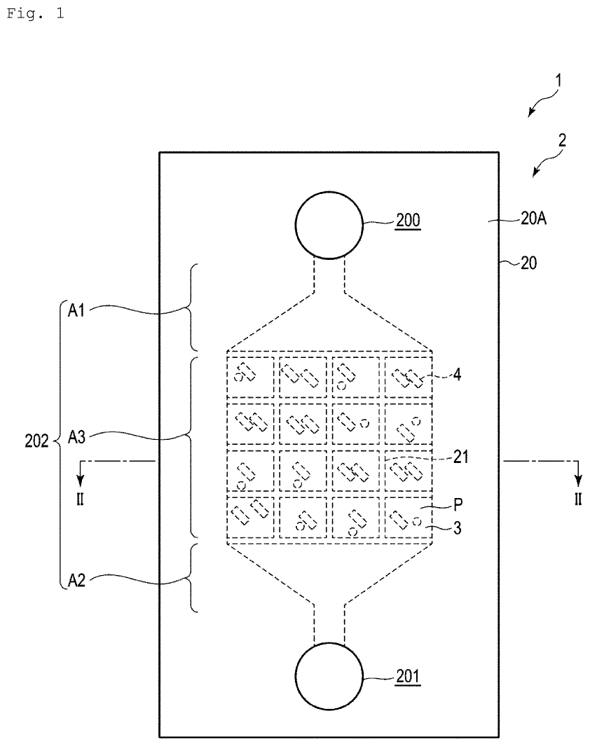

FIG. 1 is a schematic diagram showing an example of a top view of a device for observing microorganisms according to an embodiment of the present invention.

A device for observing microorganisms 1 includes a device main body 2, a medium 3, and a plurality of types of microorganisms 4.

The device main body 2 includes a culture vessel 20 having a hollow structure and a partition wall 21 provided inside the culture vessel 20.

The culture vessel 20 includes one or more inflow holes 200 and an outflow hole 201 which allow an internal space 202 and an external space of the culture vessel 20 to communicate with each other. Also, the culture vessel 20 has light-transmitting portions having light-transmitting properties at positions of a plurality of compartments. Here, although the whole culture vessel 20 is light transmissive, the culture vessel 20 may have optical transparency only at the positions of the plurality of compartments. A plurality of compartments will be described later.

The internal space 202 includes a first portion A1 communicating with one or more outflow holes 201, a second portion A2 communicating with the outflow hole, and a third portion A3 communicating the first portion A1 with the second portion A2.

The partition wall 21 partitions the internal space 202 of the culture vessel 20, specifically, the third portion A3, into a plurality of compartments P. Here, the partition walls 21 are grid-shaped. The partition wall 21 allows transfer of liquid between each of the plurality of compartments P and one or more inflow holes 200 and between each of the plurality of compartments P and the outflow hole 201. Moreover, the partition wall 21 prevents the plurality of types of microorganisms 4 located in a certain compartment from moving to another compartment.

A plurality of compartments P shown in FIG. 1 form one or more rows each composed of a plurality of compartments P arranged in a first direction. Furthermore, the one or more inflow holes 200, the one or more rows, and the outflow hole 201 are arranged in this order in the second direction intersecting the first direction.

The medium 3 fills each of the plurality of compartments P. The medium 3 is, for example, a liquid for culturing a plurality of types of microorganisms 4.

The plurality of types of microorganisms 4 are located in the internal space 202. The plurality of types of microorganisms 4 are, for example, soil microorganisms.

At least one of the plurality of compartments P includes two or more of the plurality of types of microorganisms 4 and is free from one or more of the plurality of types of microorganisms 4.

For example, two or three microorganisms exist in each of at least one of the plurality of compartments P.

In addition, it is preferable that the combination of a plurality of types of microorganisms 4 located in a certain compartment P among a plurality of compartments P be different from the combination of a plurality of types of microorganisms 4 located in at least one other compartment P.

Note that at least one of the plurality of compartments P may not include the plurality of types of microorganisms 4. Moreover, a plurality of types of microorganisms 4 may exist in the partition wall 21.

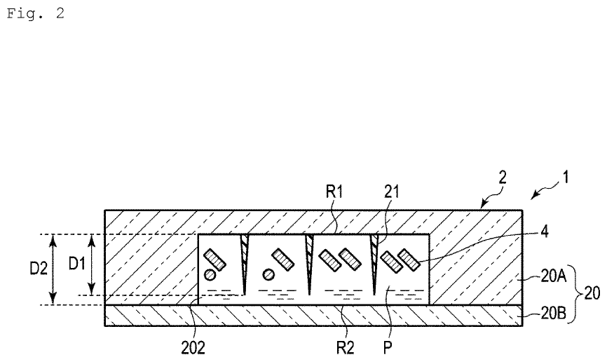

FIG. 2 is a cross-sectional view of the device for observing microorganisms shown in FIG. 1 along line II-II.

As shown in FIG. 2, the culture vessel 20 includes a first substrate 20A and a plate-like second substrate 20B bonded to the first substrate 20A. A main surface of the first substrate 20A on the side facing the second substrate 20B has two through-holes and a recess which communicates the through-holes. The two through-holes and recesses correspond to the inflow hole 200, the outflow hole 201, and the internal space 202, respectively.

The inner surface of the culture vessel 20 includes a first region R1 and a second region R2 facing each other. Furthermore, the partition wall 21 extends from the first region R1 toward the second region R2 and is spaced from the second region R2. Thus, since the partition wall 21 is spaced from the second region R2, in the device for observing microorganisms 1 shown in FIG. 1, a liquid can be transferred between each of the plurality of compartments P and one or more inflow holes 200 and between each of the plurality of compartments P and the outflow hole 201. In addition, the partition wall 21 is spaced from the second region R2 to such an extent that the plurality of types of microorganisms 4 cannot pass through between the partition wall 21 and the second region R2.

D1 shown in FIG. 2 is a length of the partition wall 21. Furthermore, D2 shown in FIG. 2 is a height of the internal space 202. A difference D2−D1 is preferably within the range of 500 nm to 1000 nm.

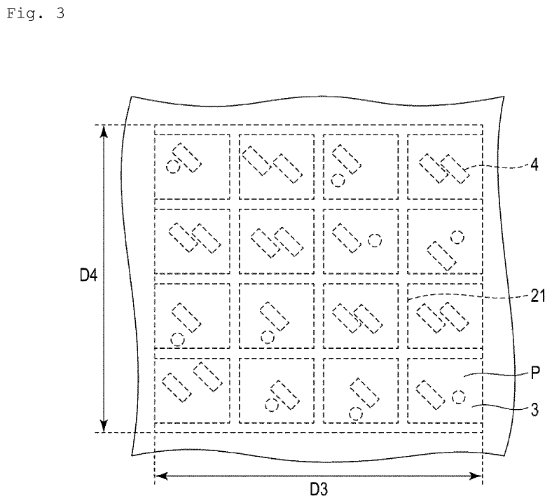

FIG. 3 is an enlarged view of the device for observing microorganisms 1 shown in FIG. 1.

D3 shown in FIG. 3 is a width of the third portion A3. The width D3 is preferably within the range of 100 μm to 10000 μm.

D4 shown in FIG. 3 is a vertical width of the third portion A3. The vertical width D4 is preferably within the range of 100 μm to 10000 μm.

The horizontal width of each compartment P is preferably within the range of 10 μm to 100 μm.

Moreover, the vertical width of each compartment P is preferably within the range of 10 μm to 100 μm.

### 2. Method for Producing Device for Observing Microorganisms

The device for observing microorganisms 1 shown in FIG. 1 can be produced using, for example, the following method.

In this method, first, an internal space 202 and one or more inflow holes 200 and outflow hole 201 each communicating the internal space 202 to the external space are provided and a culture vessel 20 having a light-transmissive portion is prepared. Here, it is assumed that the whole culture vessel 20 having a light-transmissive portion is prepared.

Subsequently, a microorganism-containing liquid including a plurality of types of microorganisms 4, a photocurable resin, and a medium is supplied to the internal space 202.

The amount of the plurality of types of microorganisms 4 included in the microorganism-containing liquid can be adjusted in accordance with, for example, the number of microorganisms located in each compartment P.

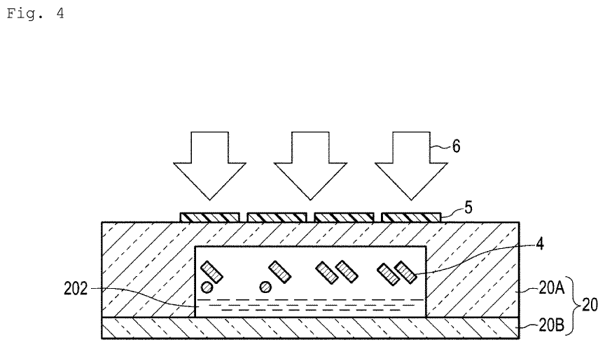

Subsequently, as shown in FIG. 4, a mask 5 is placed on or above an upper surface of the culture vessel 20. The mask 5 has a light shielding portion corresponding to the internal space 202 and a transmitting portion corresponding to the partition wall 21.

Subsequently, as shown in FIG. 4, a part of the microorganism-containing liquid is irradiated with ultraviolet rays 6 through the mask 5. The white arrow shown in FIG. 4 indicates a propagation direction of the ultraviolet rays 6.

The photocurable resin is cured through this ultraviolet irradiation only in the portion of the microorganism-containing liquid irradiated with the ultraviolet rays 6. The partition wall 21 which is made of a cured product of a photocurable resin and divides the internal space 202 into a plurality of compartments P is formed through this irradiation. The partition wall 21 is formed so that a liquid is allowed to move between each of the plurality of compartments P and one or more inflow holes 200 and between each of the plurality of compartments P and the outflow hole 201, and so that at least one of the plurality of compartments P nclude two or more of the plurality of types of microorganisms 4 and is free from one or more of the plurality of types of microorganisms 4.

Here, as shown in FIG. 2, the partition wall 21 is formed to extend from the first region R1 toward the second region R2 and is spaced from the second region R2. As described with reference to FIG. 2, the partition wall 21 is spaced from the second region R2 to such an extent that the plurality of types of microorganisms 4 cannot pass through between the partition wall 21 and the second region R2 of the inner surface of the culture vessel 20. Such partition walls 21 can be formed, for example, by adjusting the conditions of light irradiation. For example, if the irradiation time is relatively short, the curing of the photocurable resin is likely to progress on the first region R1 side and the curing of the photocurable resin is difficult to progress on the second region R2 side. Thus, a partition wall 21 as shown in FIG. 2 can be formed.

The method for producing the device for observing microorganisms 1 shown in FIG. 1 has been described above.

### 3. Culturing Method and Observing Method

According to an example, the device for observing microorganisms 1 described above can be used in a method for culturing microorganisms. A method for culturing microorganisms includes, for example, continuously or intermittently supplying a medium through one or more inflow holes 200 described above to culture a plurality of types of microorganisms 4. When the medium is supplied continuously or intermittently, the composition of the medium may be changed over time.

In addition, according to another example, the device for observing microorganisms 1 described above can be used for a microorganism observation method. The method for observing microorganisms includes, for example, observing microorganisms located in one or more of the plurality of compartments P through the light-transmitting portion, the microorganisms are two or more of the plurality of types of microorganisms 4 provided in the device for observing microorganisms 1.

The culture and observation of microorganisms described above can also be performed simultaneously.

### 4. Modification Example

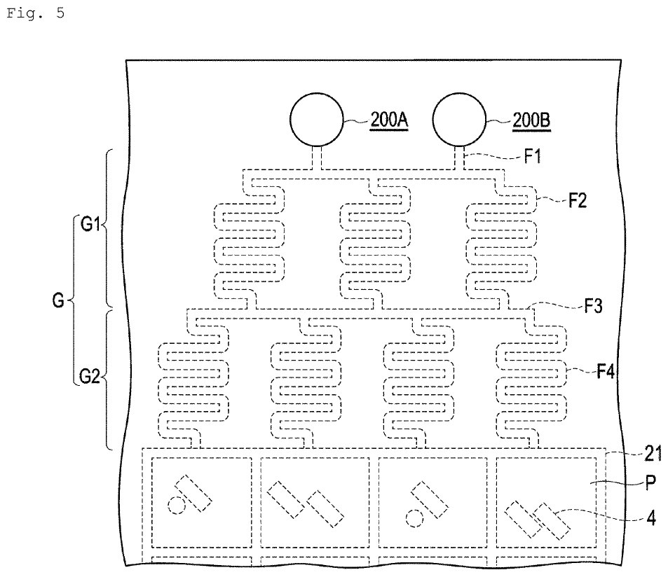

FIG. 5 is a schematic diagram showing an example of a top view of a device for observing microorganisms 1 according to another embodiment. In the device for observing microorganisms 1 shown in FIG. 5, a culture vessel 20 includes two inflow holes 200, that is, an inflow hole 200A and an inflow hole 200B. Also, a concentration gradient generation portion G is provided in the first portion A1 of the device main body 2 shown in FIG. 5, that is, in the internal space 202, which is between the inflow holes and the array composed of the plurality of compartments P. The device for observing microorganisms 1 shown in FIG. 5 is the same as the device for observing microorganisms 1 shown in FIG. 1, except that the culture vessel 20 includes two inflow holes and the device main body 2 includes the concentration gradient generation portion G.

The concentration gradient generation portion G shown in FIG. 5 is composed of a first concentration gradient generation portion G1 and a second concentration gradient generation portion G2.

The first concentration gradient generation portion G1 includes two branched first flow paths F1 and three second flow paths F2. The inlets of the two first flow paths F1 are connected to the inflow hole 200A and the inflow hole 200B, respectively. Each of the second flow path F2 meanders. Two of the three second flow paths F2 each have their inlet connected to one of the outlets that one of the two first flow paths F1 has. Also, the rest of the three second flow paths F2 have the inlets connected to one of the outlets that one of the two first flow paths F1 has and one of the outlets that the other has.

The second concentration gradient generation portion G2 includes three branched third flow paths F3 and four fourth flow paths F4. The three third flow paths F3 have inlets connected to the outlets of the three second flow paths, respectively. Each of the fourth flow path F4 meanders. Two of the four fourth flow paths F4 each have their inlet connected to one of the outlets that one of the three third flow paths F3 has. Also, the rest of the four fourth flow paths F4 have inlets connected to one of the outlets one of the two of the three third flow paths F3 has and one of the outlets the other has. The outlets of the four fourth flow paths F4 are connected to the plurality of compartments P, respectively.

In the device for observing microorganisms 1 shown in FIG. 5 described above, mediums having different compositions can be supplied to the four compartments arranged in the horizontal direction in FIG. 5 by supplying mediums having different compositions to the inflow hole 200A and the inflow hole 200B. For example, when a high-concentration medium is supplied to the inflow hole 200A and a low-concentration medium is supplied to the inflow hole 200B, the concentration of the medium can be decreased from the left compartment to the right compartment.

Note that, although the first concentration gradient generation portion G1 described above includes two first flow paths F1 and three second flow paths F2, the first concentration gradient generation portion G1 may include three or more first flow paths F1 and four or more second flow paths F2. Also, the second concentration gradient generation portion G2 may be omitted.

The device for observing microorganisms 1 shown in FIG. 5 has been described above.

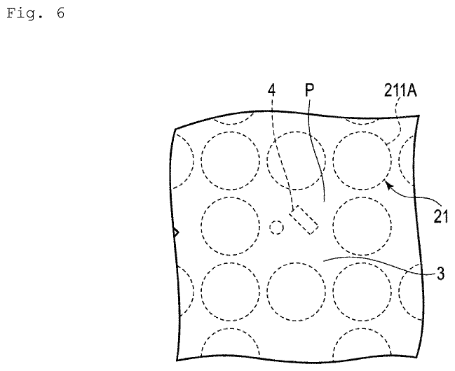

FIG. 6 is a schematic diagram showing an example of a top view of a device for observing microorganisms according to still another embodiment. The device for observing microorganisms 1 shown in FIG. 6 is the same as device for observing microorganisms 1 shown in FIG. 1, except that a shape of partition wall 21 is different from a shape of partition wall 21 shown in FIG. 1.

The partition wall 21 shown in FIG. 6 is composed of an assembly of a plurality of pillars 211A. Here, each of the pillars 211A is cylindrical. Each of the pillars 211A extends from the first region R1 to the second region R2. That is to say, although the partition wall 21 shown in FIG. 1 is spaced from the second region R2, the pillar 211A shown in FIG. 6 is in contact with the second region R2. A gap through which a liquid is allowed to move between each of the plurality of compartments P and the inflow hole 200 and between the plurality of compartments P and the outflow hole 201 is provided between two adjacent pillars 211A. A size of this gap is set to a size which does not allow movement of microorganisms.

The device for observing microorganisms 1 shown in FIG. 6 has been described above.

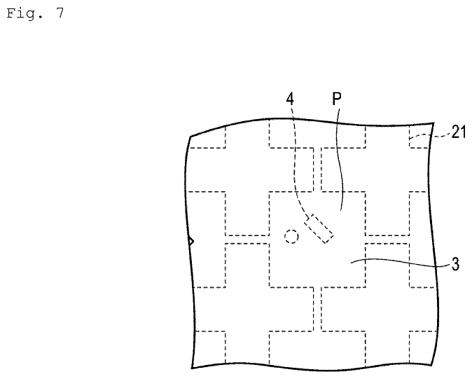

FIG. 7 is a schematic diagram showing an example of a top view of a device for observing microorganisms according to still another embodiment. The device for observing microorganisms 1 shown in FIG. 7 is the same as the device for observing microorganisms 1 shown in FIG. 1, except that a shape of the partition wall 21 is different from a shape of the partition wall 21 shown in FIG. 1.

The partition wall 21 shown in FIG. 7 extends from the first region R1 to the second region R2. That is to say, although the partition wall 21 shown in FIG. 1 is spaced from the second region R2, the partition wall 21 shown in FIG. 7 is in contact with the second region R2. Furthermore, the partition wall 21 shown in FIG. 7 includes flow paths through which a liquid is allowed to move between the plurality of compartments P and the inflow hole 200 and between each of the plurality of compartments P and the outflow hole 201. A size of this flow path is set to a size in which movement of microorganisms is not allowed.

The device for observing microorganisms 1 shown in FIG. 7 has been described above.

### 5. Effects

Incidentally, as an example of a method for co-cultivating a plurality of types of microorganisms, there is a method for culturing a plurality of types of microorganisms in a culture vessel without compartments. However, when observing a plurality of types of microorganisms cultured using this method, it was necessary to transfer a plurality of types of microorganisms from the culture vessel to the observation plate. For this reason, such a method requires time and effort during observation. In addition, when there are many types of microorganisms, it is particularly troublesome to observe them.

On the other hand, the device for observing microorganisms 1 described above has a light-transmitting portion having light-transmitting properties at the positions of a plurality of compartments P. For this reason, the microorganisms 4 located in one or more of the plurality of compartments P among the plurality of types of microorganisms 4 provided in the device for observing microorganisms 1 described above can be observed through the light-transmitting portion. As described above, according to the device for observing microorganisms 1 described above, it is possible to observe the symbiotic relationships of a plurality of combinations of microorganisms.

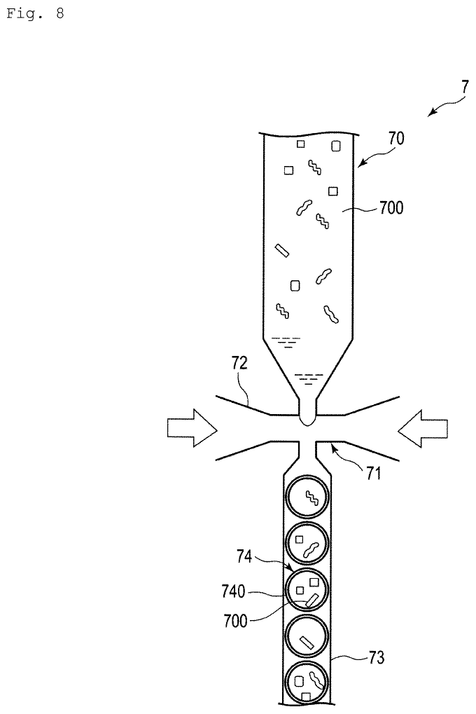

Furthermore, as another example of a method for co-cultivating a plurality of types of microorganisms, there is a method using a droplet generation device. The droplet generation device will be described below with reference to FIG. 8.

FIG. 8 is a schematic diagram showing an example of the droplet generation device 7. The droplet generation device 7 includes a culture supply portion 70, a droplet generation portion 71, an oil phase material supply portion 72, and a droplet receiving portion 73.

The culture supply portion 70 includes a culture 700 containing a plurality of types of microorganisms. The culture supply portion 70 is connected to the droplet generation portion 71. The culture supply portion 70 can intermittently supply the culture 700 to the droplet generation portion 71.

The oil phase material supply portion 72 is connected to the droplet generation portion 71. The oil phase material supply portion 72 can supply the oil phase material to the droplet generation portion 71. The white arrow shown in FIG. 8 indicates a direction in which the oil phase material supply portion 72 supplies the oil phase material to the droplet generation portion 71.

The droplet generation portion 71 is connected to the culture supply portion 70, the oil phase material supply portion 72, and the droplet receiving portion 73. In the droplet generation portion 71, the culture 700 supplied from the droplet generation portion 71 is encapsulated in the oil phase material supplied to the droplet generation portion 71. This encapsulation generates a droplet 74 composed of a culture 700 and an oil phase 740 coating the culture 700.

The droplet receiving portion 73 is a container which stores the droplets 74 generated using the droplet generation portion 71. The droplet receiving portion 73 is filled with an aqueous medium.

The droplet generation device 7 described above generates droplets 74 using the following method.

First, the culture 700 is intermittently supplied from the culture supply portion 70 to the droplet generation portion 71. Subsequently, the culture 700 supplied to the droplet generation portion 71 passes through the droplet generation portion 71. In addition, when the culture 700 enters the droplet receiving portion 73, the microorganism medium 700 is encapsulated in the oil phase material supplied to the droplet generation portion 71 using the oil phase material supply portion 72. A droplet 74 is generated through this encapsulation.

The method for generating the droplet 74 has been described above.

A plurality of types of microorganisms can be co-cultivated in the droplets obtained using the droplet generation device 7 described above. For example, it is possible to co-cultivate two or three microorganisms in one droplet. Such methods are appropriate for co-cultivating each of a large number of combinations composed of a plurality of types of microorganisms. However, in such a droplet, it is difficult for a substance such as a medium to move between the inside and the outside of the droplet. For this reason, microorganisms in the droplet stop growing when the nutrients in the droplet are depleted. Therefore, it is difficult to culture and observe microorganisms in droplets for a long period of time. Also, according to the droplet generation device 7 described above, the composition of the medium cannot be changed for each droplet. For this reason, if different droplets require different substances for survival, symbiotic relationships between a plurality of types of microorganisms cannot be observed in some droplets.

On the other hand, in the device for observing microorganisms 1 described above, the partition wall 21 allows transfer of a liquid between each of the plurality of compartments P and one or more inflow holes 200 and between each of the plurality of compartments P and the outflow hole 201. Therefore, it is possible to continuously or intermittently supply a liquid such as the medium 3 to the plurality of compartments P. Therefore, in the device for observing microorganisms 1 described above, it is possible to culture a plurality of types of microorganisms 4 for a long period of time. It can be seen that, in a compartment P which has been successfully cultured using the above-described method for culturing microorganisms, a plurality of types of microorganisms 4 located in the compartment P can coexist with each other.

Furthermore, if using the device for observing microorganisms 1 including the concentration gradient generation portion G described above, the concentration of the medium which fills one compartment P and the medium which fills another compartment P can be different. As described above, according to the device for observing microorganisms 1 including the concentration gradient generation portion G described above, the composition of the medium can be changed in accordance with the combination of microorganisms.

As described above, with the device for observing microorganisms 1 described above, it is possible to culture a plurality of types of microorganisms 4 for a long period of time, that is, to continuously culture them. Furthermore, as described above, according to the device for observing microorganisms 1 described above, it is possible to observe the symbiotic relationships of a plurality of combinations of microorganisms. For this reason, according to the device for observing microorganisms 1 described above, it is possible to continuously culture and observe various combinations of microorganisms.

Furthermore, since the above-described device for observing microorganisms 1 includes the above-described partition wall 21, it is possible to change the type of medium which fills the compartment P over time.

### REFERENCE SIGNS LIST

1 Device for observing microorganisms
2 Device main body
3 Medium
4 Microorganism
5 Mask
6 Ultraviolet rays
7 Droplet generation device
20 Culture vessel
20A First substrate
20B Second substrate
21 Partition wall
70 Culture supply portion
71 Droplet generation portion
72 Oil phase material supply portion
73 Droplet receiving portion
74 Droplet
200 Inflow hole
200A Inflow hole
200B Inflow hole
201 Outflow hole
202 Internal space
211A Pillar
700 Culture
740 Oil phase
A1 First portion
A2 Second portion
A3 Third portion
F1 First flow path
F2 Second flow path.
F3 Third flow path.
F4 Fourth flow path.
G Concentration gradient generation portion
G1 First concentration gradient generation portion.
G2 Second concentration gradient generation portion
P Compartment
R1 First region
R2 Second region

1. A device for observing microorganisms comprising:
a device main body including a culture vessel having a hollow structure and a partition wall provided in the culture vessel and partitioning an internal space of the culture vessel into a plurality of compartments, the culture vessel having one or more inflow holes and an outflow hole each communicating the internal space to an external space, the culture vessel including a light-transmitting portion having light-transmitting properties at positions of the compartments, and the partition wall allowing transfer of liquid between each of the plurality of compartments and the one or more inflow holes and between each of the plurality of compartments and the outflow hole:
a medium filling each of the plurality of compartments; and
a plurality of types of microorganisms located in the internal space,
wherein at least one of the plurality of compartments includes two or more of the plurality of types of microorganisms and is free from one or more of the plurality of types of microorganisms.

2. The device for observing microorganisms according to claim 1, wherein an inner surface of the culture vessel includes a first region and a second region facing each other and the partition wall extends from the first region toward the second region and is spaced from the second region.

3. The device for observing microorganisms according to claim 1, wherein the one or more inflow holes are two or more inflow holes,
the device main body is provided with a concentration gradient generation portion between the two or more inflow holes and an array composed of the plurality of compartments in the internal space, the concentration gradient generation portion including two or more first flow paths each being branched and three or more second flow paths,
the two or more first flow paths have inlets connected to the two or more inflow holes, respectively,
each of two of the three or more second flow paths has an inlet connected to one of the outlets of one of the two or more first flow paths, and
each of the rest of the three or more second flow paths has an inlet connected to one of the outlets of one of a two of the two or more first flow paths and connected to one of the outlets of the other one of the two of the two or more first flow paths.

4. The device for observing microorganisms according to claim 1, wherein the plurality of compartments form one or more rows each composed of a plurality of compartments arranged in the first direction, and the one or more inflow holes, the one or more rows, and the outflow hole are arranged in this order in a second direction intersecting the first direction.

5. A method for producing device for observing microorganisms, comprising:
preparing a culture vessel including an internal space, one or more inflow holes and an outflow hole each communicating the internal space to an external space, and the culture vessel having a light-transmitting portion having light-transmitting properties:
supplying a microorganism-containing liquid containing a plurality of types of microorganisms, a photocurable resin, and a medium to the internal space; and
forming a partition wall made of a cured product of the photocurable resin and partitioning the internal space into a plurality of compartments by irradiating a part of the microorganism-containing liquid with light through the light-transmitting portion so that a transfer of a liquid is allowed between each of the plurality of compartments and the one or more inflow holes and between each of the plurality of compartments and the outflow hole, and so that at least one of the plurality of compartments includes two or more of the plurality of types of microorganisms and is free from one or more of the plurality of types of microorganisms.

6. The method for producing device for observing microorganisms according to claim 5, wherein an inner surface of the culture vessel includes a first region and a second region facing each other and the partition wall is formed to extend from the first region toward the second region and is spaced from the second region.

7. A method of culturing microorganisms, comprising:
continuously or intermittently supplying the medium to the device for observing microorganisms according to claim 1 through the one or more inflow holes to culture the plurality of types of microorganisms.

8. A method of observing microorganisms, comprising:
observing microorganisms located in one or more of the plurality of compartments through the light-transmitting portion, the microorganisms are two or more of the plurality of types of microorganisms included in the device for observing microorganisms according to claim 1.
